(19) United States
(12) Patent Application Publication
(10) Pub. No.: US 2025/0025192 A1
(43) Pub. Date: Jan. 23, 2025

(54) CATHETER WITH LUBRICIOUS COATING FOR DISTAL ACCESS TO THE VASCULATURE INCLUDING THE NEUROVASCULATURE

(71) Applicant: Julier Medical SAS, Paris (FR)

(72) Inventor: Niall BEHAN, Dubendorf (CH)

(73) Assignee: Julier Medical SAS, Paris (FR)

(21) Appl. No.: 18/776,231

(22) Filed: Jul. 17, 2024

Related U.S. Application Data
(60) Provisional application No. 63/613,448, filed on Dec. 21, 2023.
(60) Provisional application No. 63/514,535, filed on Jul. 19, 2023.

(30) Foreign Application Priority Data
Jul. 18, 2023 (EP) 23306235.5

(51) Int. Cl. A61B 17/221; A61B 17/00

(52) U.S. Cl. CPC A61B 17/221; A61B 17/00234; A61B 2017/00238; A61B 2017/00305; A61B 2017/00367; A61B 2017/00477; A61B 2017/00526; A61B 2017/00845; A61B 2017/00853; A61B 2017/00862; A61B 2017/00867; A61B 2017/00938; A61B 2017/2215

(57) ABSTRACT

Systems and methods are provided for applying a lubricious coating to a catheter such as a catheter for distal access and/or removing an obstruction from or clot from a blood vessel, e.g., in the neurovasculature. A lubricious coating may be applied to an inner and outer surface of the elongated tube of the catheter. For example, an expandable braid at a distal portion of the catheter may be coated on its inner and outer surfaces to facilitate advancement through the vasculature and slidability of a catheter shaft within the expandable braid.

### CROSS-REFERENCE TO RELATED APPLICATIONS

This application claims priority to U.S. Provisional Patent Application Ser. No. 63/613,448, filed Dec. 21, 2023, U.S. Provisional Patent Application Ser. No. 63/514,535, filed Jul. 19, 2023, and EP patent application Ser. No. 23/306,235.5, filed Jul. 18, 2023, the entire contents of each of which are incorporated herein by reference.

### TECHNICAL FIELD

This technology relates, in general, to systems and methods for applying a lubricious coating to a catheter system, for example, a catheter for distal access and/or for removing an obstruction from a blood vessel such as thrombus, including from the neurovasculature.

### BACKGROUND

Ischemic stroke is caused by a partial or complete interruption to cerebral blood perfusion. Such an interruption may be caused by a thrombus or embolus, i.e. a clot, originating from a more proximal location within the bloodstream, becoming trapped within the narrowing intracranial vessels. The interruption of blood flow to a portion of the brain for any prolonged period of time results in a region of infarcted tissue, known as the core infarct, that is irreversibly damaged and grows larger with time. Infarcted regions of the brain will result in neurological deficits that may range from minor speech and coordination problems to total loss of muscle and cognitive control.

The oxygen-starved region around the core infarct also grows larger the longer the interruption continues. This region, known as the penumbra, may regenerate if blood perfusion is restored in a timely manner. This phenomenon of a treatable ischemic event has given rise to the phrase “time is brain,” now common amongst associated clinicians.

In recent years, the technology for mechanical removal of such blockages has enabled reperfusion of blood flow and effective treatment of stroke in some cases. Within recent years, the first of several clinical studies were published that validated the efficacy of stent retrievers for blood flow restoration versus the standard of care at the time, which was intravenous thrombolysis medication and aspiration clot retrieval

Mechanical clot retrieval devices are generally metal baskets or stents that are connected to a retrieval wire. During a clot removal procedure, a guide wire is placed across the length of the clot and a catheter is navigated over the guidewire to cross the clot. The clot retrieval device is delivered through the catheter to the required location. The catheter or sheath is retracted from over the clot retrieval device, which then expands and engages with the clot. The clot retrieval device and the clot integrated therein can then be removed through the blood vessel using tension by pulling the retrieval wire. Optionally, a suction catheter can be used to help with removal

In many cases, the clot cannot be removed intact during the first pass of the clot retrieval device and multiple passes are required to get blood flow restoration. The improvement in first pass clot removal is a target of many current developments in this field of technology.

In practice, the clinician will use several different tools during the endovascular procedure to remove the clot. Generally, a guidewire will be placed into the femoral artery using the modified Seldinger technique and will be navigated through the carotid artery into the cerebral vasculature of the brain.

The guidewire is then pushed through the clot. Once the guidewire is in place, a very narrow tubular catheter known as a microcatheter (approx. 0.4 mm diameter) is advanced to the distal side of the clot over the wire.

The guidewire is then removed and a stent retriever is pushed through the microcatheter and deployed along the length of the clot. The stent retriever engages with the clot and is then retracted to remove the clot from the circulatory system of the patient. In most cases, this procedure is carried out while simultaneously applying aspiration through a larger diameter catheter that is navigated close to the clot over the microcatheter. The aspiration catheter is stiffer than the microcatheter due to its larger diameter and reinforcement, which is required to prevent collapse during suction. The microcatheter is therefore also required as a support and guide for introduction of the aspiration catheter through the vasculature.

European Patent Application Pub. No. EP 3718492 A1 discloses a catheter apparatus for the removal of a clot from the circulatory system of a patient in which a plurality of clot-engaging elements are deployable independently from each other. In this way, the first pass clot removal rate can be improved.

In addition to the above technique, there have been multiple different approaches described using the combination of stent retriever and aspiration catheter. Some of these techniques describe the complete withdrawal of stent retriever and clot into the aspiration catheter. Other approaches are directed to the withdrawal of the clot and stent retriever using the aspiration catheter, wherein the proximal part of the clot is attached to the aspiration catheter during the removal.

The clot retrieval approach chosen is often influenced by the clot composition and in some cases where the clot is very soft thrombus, an aspiration catheter alone may be sufficient to remove the entire clot via suction. In this case, a microcatheter is still required for support to help with navigation of the aspiration catheter to the desired site.

Regardless of the specific technique used, removal of the clot without delay is crucially important. Although the guidewire and microcatheter are generally advanced to the clot quickly, the positioning of the aspiration catheter may be a limiting factor. The process of advancing the aspiration catheter becomes particularly difficult after passing through the internal carotid artery. This is due to the narrow and tortuous vessels after this point in the cerebral vasculature and is exacerbated in older patients where the vasculature is diseased and elongated. This time-consuming part of the procedure may impact the patient's clinical outcome. In the case where the clot is not removed during the first pass, the cumulative time taken during multiple attempts can be significant.

Devices have been described with a distally expanding funnel at the distal end of the aspiration catheter. These devices are generally separate to the aspiration catheter and are fed through the lumen and pushed out of the distal end of the aspiration catheter to expand. Such devices are intended to widen the already large luminal diameter of the aspiration catheter and to engulf a withdrawing stent retriever and/or clot to ensure no microemboli are released during the clot removal. WO 02/087677 A2, U.S. Patent App. Pub. No. 2017/0303949 to Ribo Jacobi, WO 2016/113047 A1, U.S. Patent App. Pub. No. 2019/0269491 to Jalgaonkar, U.S. Patent App. Pub. No. 2017/0333060 to Panian are examples of documents directed to this kind of technique.

U.S. Pat. No. 6,632,236 to Hogendijk describes apparatus for occluding a vessel and enhancing blood flow within a catheter. The catheter includes a multi-section self-expanding wire weave forming a radially expandable body and an occlusive distal section, covered with an elastomeric polymeric coating, and disposed within an outer sheath.

U.S. Pat. No. 6,929,634 to Dorros describes apparatus and methods for treatment of stroke using a catheter having a distal occlusive member in the common carotid artery of the hemisphere of the cerebral occlusion. Retrograde flow is provided through the catheter to effectively control cerebral flow characteristics. Under such controlled flow conditions, a thrombectomy device is used to treat the occlusion, and any emboli generated are directed into the catheter.

U.S. Pat. No. 6,206,868 to Parodi discloses an occlusive element with a self-expanding wire mesh basket covered with an elastomeric polymer coating. The catheter is initially surrounded by a movable sheath, and is inserted transluminally with the sheath at a distalmost position. The sheath is retracted proximally to cause the basket to deploy, and the basket is again collapsed within the sheath by moving the sheath to its distal-most position.

U.S. Patent App. Pub. No. 2017/0238951 to Yang describes a neurovascular catheter for distal neurovascular access or aspiration. The catheter includes an elongate flexible tubular body, having a proximal end, a distal end, and a side wall defining a central lumen. A distal zone of the tubular body includes a tubular inner liner, a tie layer separated from the lumen by the inner liner, a helical coil surrounding the tie layer, an outer jacket surrounding the helical coil, and an opening at the distal end. Adjacent windings of the helical coil are spaced progressively further apart in the distal direction. The opening at the distal end of the tubular body is enlargeable from a first inside diameter for transluminal navigation to a second, larger inside diameter to facilitate aspiration of thrombus into the lumen.

U.S. Patent App. Pub. No. 2017/0143938 to Ogle describes a suction catheter system is described with a suction nozzle that can extend from a guide catheter of the like. The suction nozzle can be positioned by tracking the suction nozzle through a vessel while moving a proximal portion of the suction extension within the lumen of the guide catheter. A suction lumen extends from the proximal end of the guide catheter through at least part of the guide catheter central lumen and through the suction tip.

U.S. Patent App. Pub. No. 2016/0256180 to Vale describes a rapid exchange (RX) catheter that provides a proximal seal against a guide catheter inner lumen so that aspiration may be applied through a guide catheter. The catheter may include an exit port that defines a transfer port for aspiration and may enable minimal frictional engagement with the guide catheter proximal of the exit port. Aspiration can be applied to the lumen of the guide catheter and may be directed to and effective at the tip of the RX aspiration catheter. A tip of the RX catheter may facilitate aspiration and retrieval of the clot by expanding under load and can also partially or fully occlude the vessel.

WO 2017/097616 A1 discloses a plurality of devices and methods for removing blockages from blood vessels. A stent retriever is first deployed via a microcatheter and, to improve the clot removal process, an aspiration catheter is then advanced to the position of the clot. A clot receptor device is deployed, which circumferentially seals against a distal section of the aspiration catheter, such that the stent retriever and the clot may be aspirated through the tapered opening of the receptor device during the removal process. The stent retriever may also deployed using the microcatheter and an aspiration catheter is then forwarded to the position of the clot to aspirate the stent retriever and the clot.

U.S. Pat. No. 8,425,549 to Lenker discloses a catheter having a distal portion, which can be radially expanded by means of a coil or a helical ribbon that is distally displaceable within the catheter. The expanded configuration allows applying a negative pressure through the lumen of the catheter to aspirate obstructive matter through the distal end opening and into the lumen of the catheter.

Further devices for neurovascular endoluminal intervention of the kind as indicated are disclosed in WO 2016/126974, WO 2018/169959, and WO 98/23320.

Besides the above-described catheter devices, introducer sheaths are known, which are short cannula-like devices that are used for vessel access. They are inserted into the target vessel percutaneously and a central dilator is then removed to allow access for insertion of other devices such as guidewires and catheters. Recently a number of introducer devices have been developed that have the capability to expand to accommodate devices larger than the nominal vessel size. Examples of expanding sheath type devices are the Edwards eSheath™ and the Terumo Solopath™.

Due to the criticality of time with respect to oxygen, it is paramount that the catheter is expediently navigated to the target, e.g., the site of the clot and/or thrombus. However, friction between the catheter and the patient's vasculature can resist advancement of the catheter as it traverses the patient's vasculature. Further, friction between catheter components (e.g., friction between the microcatheter and the stent retriever) can further delay retrieval and removal of the clot and/or thrombus. Traditional lubricious coatings are formed from combinations of UV cured acrylic monomers and hydrophilic compounds that are generally quite stiff and non-elastic. In addition, UV curing is used on an outer surface of a tube, but would be technically challenging on an inner lumen.

Accordingly, there is a need for low friction catheters and/or methods for making low friction catheters with lubricious coating for efficiently navigating the vasculature of a patient, for example, for retrieving a clot or obstruction. U.S. Pat. No. 11,622,781 to Behan and U.S. Pat. No. 11,737,767 to Behan and Grandidier, the entire contents of each of which are incorporated herein by reference, describes improved catheters for endoluminal intervention, e.g., for the treatment of ischemic stroke, that overcome many of the drawbacks of the foregoing. It would be beneficial to provide improved designs for enhancing advancement of a catheter and slidability of catheter shafts relative to one another for distal access in the vasculature and performing an intervention within the vasculature, such as obstruction removal.

### SUMMARY

Provided herein are systems and methods for applying a lubricious coating to a catheter such as a distal access catheter and/or a catheter for removing an obstruction from or clot from a blood vessel, including in the neurovasculature. For example, an improved catheter is provided that is sufficiently small and flexible to permit navigation through small and/or tortuous vessels (e.g., the neurovasculature) while being sufficiently robust to perform an intervention at the target location in the blood vessel, such as removing an obstruction such as a clot/thrombus (e.g., via aspiration through the catheter). The catheter is designed to be easily expandable for obstruction removal and collapsible for delivery and removal within the vasculature. Further, the lubricious nature of the catheter facilitates advancement and slidability of catheter shafts that are embedded and movable relative to one another at the target location.

The catheter may include at least an elongated tube having a proximal section and a distal section which may be a braided section at a distal end for expansion in the vasculature, for example, to retrieve a clot or obstruction. The distal section may form a tube and the catheter may cause the distal section to transition from a neutral position to an extended or expanded state with a diameter larger than a collapsed state. The elongated tube and the distal section may include an inner and outer surface made of silicone or polyurethane. A lubricious coating may be applied to the inner and outer surface of the elongated tube, including the distal section. The elongated tube may first be prepared by exposing the elongated tube to an initial solution such as a methyl acetate solution and/or a primer coating which may include (3-Aminopropyl)tricthoxysilane (APTES). A reactive primer solution may also optionally be applied. For example, a reactive primer solution including polyethylenimie (PEI) and perfluorophenylazide (PFPA) may be used. The catheter tube may then be exposed to a lubricious coating solution which may include a methyl ethyl ketone (MEK) solution include poly(methyl vinyl ether-maleic acide).

A method is provided herein for applying a lubricious coating to a catheter. The method may include selecting a catheter having an elongated tube including a proximal section and a braided section at a distal end, the elongated tube having an inner surface and an outer surface each including silicone or polyurethane, the catheter designed to remove an obstruction from a blood vessel, exposing a distal portion of the catheter having the braided section and at least part of the proximal section to a first solution including methyl acetate for a first duration, exposing, after exposing the distal portion of the catheter to the first solution, the distal portion of the catheter to a second solution including (3-Aminopropyl) triethoxysilane (APTES) for a second duration, exposing, after exposing the distal portion of the catheter to the second solution, the distal portion of the catheter to a lubricious coating solution including a poly(methyl vinyl ether-maleic acid) copolymer to apply a lubricous coating to the distal portion of the catheter, and heating, after exposing the distal portion of the catheter to the lubricious coating solution, the distal portion of the catheter at a first temperature for a third duration. A catheter is also provided herein, made according to the foregoing method.

The first duration may be between 22 and 26 hours, the second duration may be between 20 and 40 minutes, and the third duration may be between 35 and 55 minutes, and the first temperature may be between 90° F. to 110° C. The second solution may be an aqueous solution may include 1% weight-by-volume of (3-Aminopropyl) triethoxysilane. The lubricious coating solution may include 2% weight-by-volume of methyl ethyl ketone (MEK). The method, may include, after heating the distal portion of the catheter, exposing at least the distal portion of the catheter to a fourth solution including equal portions water and ethanol. The fourth solution may include 1 mol of NaOH and at least the distal portion of the catheter may be exposed to the fourth solution for a duration between 30 seconds and 90 seconds.

After exposing the distal portion of the catheter to the first solution, the method may include drying at least the distal end in an oven for a duration between 90 minutes and 150 minutes at a temperature between 70° C. and 90° C. After exposing the distal portion of the catheter to the second solution, the method may include one or more of exposing the distal portion of the catheter to distilled water and drying the distal portion in an oven for a duration between 30 and 90 minutes at a temperature between 70° C. and 90° C. After exposing the distal portion of the catheter to the second solution, the method may include one or more of exposing the distal portion of the catheter to distilled water and drying the distal portion of the catheter at room temperature.

Yet another method is provided herein for applying a lubricious coating to a catheter. The method may include selecting a catheter including an elongated tube having a proximal section and a braided section at a distal end, the elongated tube having an inner surface and an outer surface each including silicone or polyurethane, the catheter designed remove an obstruction from a blood vessel, exposing a distal portion of the catheter having the braided section and at least part of the proximal section to a first solution including methyl acetate for a first duration, exposing, after exposing the distal portion of the catheter to the first solution, the distal portion of the catheter to a second solution including (3-Aminopropyl) triethoxysilane (APTES) for a second duration, selecting a reactive primer solution including Polyethylenimie (PEI) reacted with an N-Hydroxysuccinimide (NHS) grafted Perfluorophenylazide (PFPA) in ethanol, exposing, after exposing the distal portion of the catheter to the second solution, the distal portion of catheter to the reactive primer solution, and, exposing, after exposing the distal portion of the catheter to the primer coating, the distal portion of catheter to a lubricious coating solution including a poly(methyl vinyl ether-maleic acid) copolymer to apply a lubricous coating to the distal end of the catheter. A catheter is also provided herein, made according to the foregoing method.

The method may include, after exposing the distal portion of the catheter to the lubricious coating solution, heating the distal portion of the catheter for a third duration between 35 and 55 minutes at a first temperature between 90° C. to 110° C. The method may include, after exposing the distal portion of the catheter to the primer coating, heating the distal portion of the catheter for a third duration between 45 and 75 minutes at a first temperature between 110° C. to 130° C. The lubricious coating solution may include 25,000 g/mol of branched Polyethylenimie (PEI). The reactive primer solution may include a ratio of PEI to PFPA between 4:1 to 10:1. The first duration may be between 22 and 26 hours and the second duration may be between 20 and 40 minutes. The second solution may be an aqueous solution including 1% weight-by-volume of (3-Aminopropyl)triethoxysilane. The lubricious coating solution including 2% weight-by-volume of methyl ethyl ketone (MEK). The method may include, after heating the distal portion of the catheter, exposing the distal portion of the catheter to a fourth solution including equal portions water and ethanol and further including 1 mol of NaOH.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1A shows exemplary components of a catheter for accessing the vasculature, e.g., for removing an obstruction(s) from a blood vessel.

FIGS. 1B and 1C show, respectively, the catheter in the collapsed state and the expanded state.

FIG. 2 shows the distal region of an elongated tube of the catheter for an exemplary embodiment having a collapsible and expandable braided section using a self-collapsing and expanding mechanism.

FIGS. 3A-3D show an exemplary process for applying an inner coating and an outer coating to an expandable portion of a catheter.

FIGS. 4A-4C illustrate cross-sectional and side views of an expandable portion of a catheter (e.g., the braid).

FIG. 5 illustrates a process flow for embodiments for applying a preliminary solution, a primer coating, and a lubricious coating.

FIG. 6 illustrates a process flow for applying a preliminary solution, a primer coating, a reactive primer coating, and a lubricious coating.

The foregoing and other features of the present invention will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION OF THE INVENTION

This technology is directed to systems and methods for applying a lubricious coating to a catheter for efficiently and expediently performing an intravascular intervention, such as removing an obstruction(s) from a blood vessel. The catheter may be a neurovascular catheter and may be used for the treatment of ischemic stroke, for example, and the lubricious coating (e.g., hydrophilic lubricious coating) may allow an overall easier and faster removal of a clot from a blood vessel. For example, the lubricous coating may become slippery when it comes into contact with a wet substance. Advantageously, the lubricious coating is designed to coat a portion of the catheter that expands and contracts. For example, the lubricious coating may cover a stretchable and expandable braid encapsulated with a biocompatible material such as a polymer or silicone. By coating inner and outer surfaces of the encapsulated braid with the lubricant, the catheter is designed to be advanced through challenging vasculature (e.g., neurovasculature for treating stroke) via the lubricated outer surface while permitting other catheter shafts to be slid into and out of the encapsulated braid via the lubricated inner surface.

The catheter may have a collapsed state where the distal outer section may be designed to be easily navigated through the vasculature including through small and/or tortuous vessels (e.g., the neurovasculature). The catheter may then, reversibly, be changed to an expanded state whereby the distal outer section is expanded to a wider diameter, which may be approximately equivalent to the diameter of the proximal outer section. This approach enables the catheter to be easily and rapidly navigated to the target site and subsequently dilated to facilitate removal of the one or several clots by aspiration. Using this design, a clinician does not waste valuable time navigating a large aspiration catheter through tortuous vessels. In addition, a microcatheter and an aspiration catheter may be combined into a single catheter, which also represents cost savings. The coatings described herein may be applied to the catheters described in U.S. Pat. No. 11,737,767 to Behan and Grandidier and/or U.S. Pat. No. 11,622,781 to Behan, the entire contents of each of which are incorporated by reference herein.

The catheter in the Behan patents has a distal expandable section that may be constructed from an elastomer coated nitinol braid. Upon expansion of the distal section in the target vessel, a reinforcing catheter in the form of a laser cut hypotube can be advanced through the lumen. Described herein is a low friction coating on the innermost lumen surface of the expandable braid section that facilitates slidability of the reinforcing catheter shaft within the expanded braided portion. The coating is lubricous and elastic and applied in a reproducible manner.

A lubricious coating may be applied to both inner and outer surfaces of an elongated tube of the catheter, which may include silicone or polyurethane inner and outer surfaces.

The lubricious, clastic coating on the distal expandable section of the catheter may be formed from a combination of soft, elastomeric polyurethanes. A polyurethane elastomer material (e.g., Tecoflex from Lubrizol) may be used to build up layers within which the braided section of the catheter is sandwiched. A combination of Shore 93A and Shore 80A may be used.

In some embodiments described herein, the elongated tube may first be exposed to an initial solution such as a methyl acetate solution and/or a primer coating which may include (3-Aminopropyl)triethoxysilane (APTES). A reactive primer coating may also optionally be applied. For example, a reactive primer coating including polyethylenimie (PEI) and perfluorophenylazide (PFPA) may be applied to the elongated tube. The catheter tube may then be exposed to a lubricious coating solution which may include a methyl ethyl ketone (MEK) solution of poly (methyl vinyl ether-maleic acide) copolymer.

The lubricious coating may chemically bound (e.g., via covalent bonding) to the device surface and/or may prevent formation of embolic particles. As cured silicone substrate is relatively inert, the surface may be chemically activated to provide reactive sites through which a coating can attach. Further, silicone elastomers often include a small amount of low molecular weight extractable compounds such as cyclic siloxanes, which may be removed prior to any bonding step. Optionally, the polymeric substrate may be treated with a plasma, corona, or etching step to enhance the chemical bond formed with a subsequently applied coating.

While the lubricious coating applied to an outer surface of the elongated tube of the catheter may facilitate efficient navigation of the catheter through a patient's neurovasculature, the lubricious coating applied to an internal surface of the elongated tube of the catheter may facilitate movement of an internal catheter or tube within the inner surface. For example, the lubricious coating may facilitate smooth advancement and retraction an the intermediate coiled reinforcement shaft.

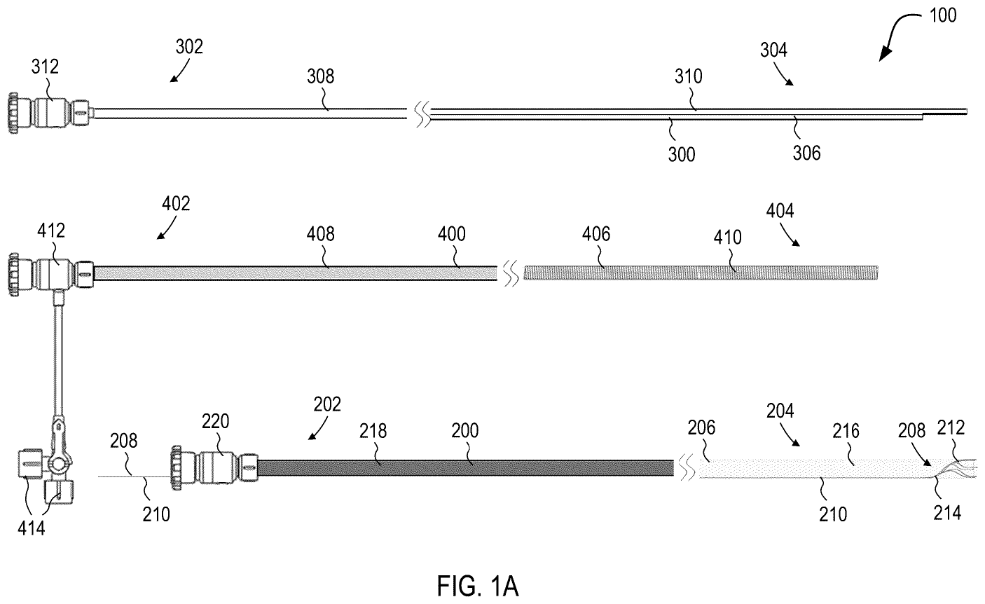

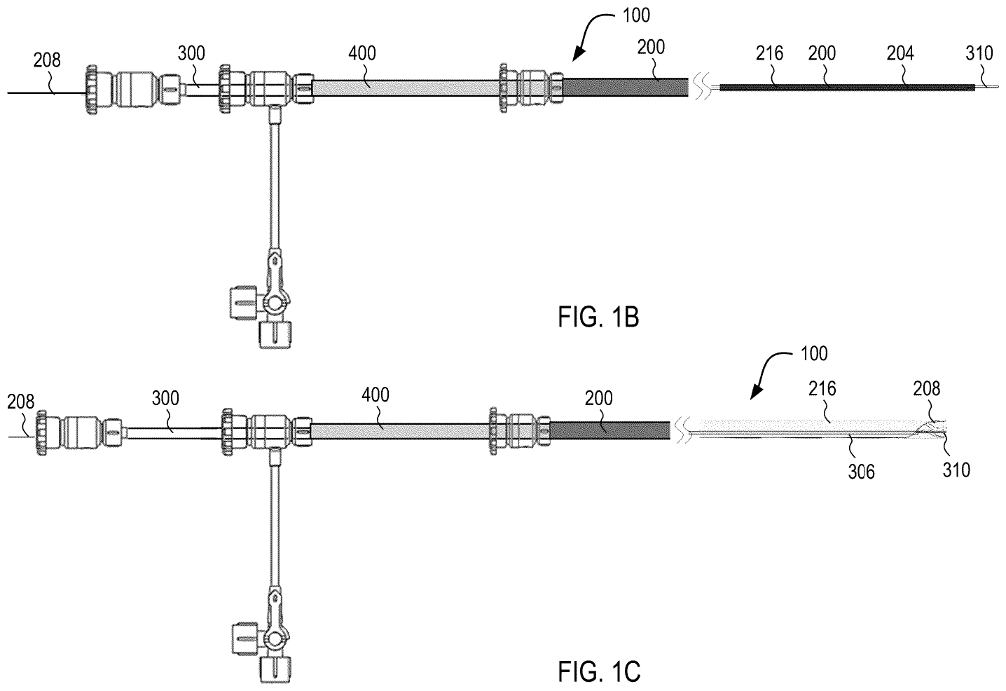

Referring now to FIG. 1A, an exemplary catheter is shown that may be used for accessing the vasculature for performing an intervention such as removing an obstruction(s) from a blood vessel, for example, to treat a stroke. Catheter 100 illustratively includes elongated tube 200 that is transitionable between an expanded state and a collapsed state. Elongated tube 200 has proximal region 202, distal region 204, and lumen 206 extending therebetween. Elongated tube 200 is sized and shaped to be advanced through the blood vessel to the target site (e.g., an obstruction(s) within the blood vessel(s)) in the collapsed state.

To transition elongated tube 200 between the collapsed state and the expanded state, actuation wire 208 is provided. As illustrated, actuation wire 208 may include elongated shaft 210 coupled to a plurality of struts 212 via articulation region 214. Each one of the plurality of struts 212 may be affixed about a circumference of the distal end of elongated tube 200. Actuation wire 208 may have a length longer than elongated tube 200. As such, actuation wire 208 may be coupled to the distal end of elongated tube and extend out the proximal end, as shown in FIG. 1A, for manipulation by the clinician.

The portion of elongated tube 200 at distal region 204 may be formed of a contractible and expandable material such as a coil, a laser-cut tube, or braid 216 as illustrated. Distal region 204 may be coated with an expandable biocompatible material such as an elastomer. For example, the collapsible/expandable portion of elongated tube 200 may be an elastomer coated braid or coil. Proximal region 202 may be formed of a different material than distal region 204. For example, proximal region 202 may include shaft 218 formed from a polymer known in the art of catheter design. The diameter of proximal region 202 may be fixed such that only distal region 204 has a variable diameter. The elastomer coated braid or coil may be bonded to proximal region 202, for example, via an adhesive. Elongated tube 200 may include hemostasis valve 220 at the proximal end to permit insertion of additional interventional devices into lumen 206 of elongated tube and to close off proximal end of lumen 206 when hemostasis valve 220 is closed.

Shaft 218 of elongated tube 200 is flexible. Distal region 204 (illustratively, braid 216) is particularly flexible to permit navigation through the vasculature, including through small and/or tortuous vessels. Shaft 218 may be made from another may be coated in polyurethane and/or silicone. Similarly, distal region 204 may be made coated in polyurethane and/or silicone such that a continuous and flexible inner and outer surface is formed at distal region 204.

Catheter 100 further may include actuator tube 300, which is sized and shaped to be disposed within elongated tube 200. Actuator tube 300 includes proximal region 302, distal region 304, and lumen 306 extending therebetween. Lumen 306 is sized and shaped to receive actuation wire 208 therethrough. Actuator tube 300 may have a length longer than the shaft of elongated tube 200, although actuation wire 208 may be longer than actuator tube 300.

Actuator tube 300 works together with actuation wire 208 to cause distal region 204 of elongated tube 200 to transition between the collapsed state and the expanded state. This provides a self-collapsing mechanism for easy and repeatable transition between these states. For example, translation of actuation wire 208 relative to actuation tube 300 causes the plurality of struts 212 to expand radially outward to transition elongated tube 200 to the expanded state, thereby permitting removal of the obstruction from the blood vessel.

Actuator tube 300 may function as a microcatheter. Shaft 308 at proximal region 302 may be relatively stiff, e.g., a hypotube. The diameter of actuator tube 300 may be fixed such that actuator tube 300 is not expandable. Distal region 304 may be more flexible than proximal region to permit bending and navigation through tortuous vessels. Actuator tube 300 may include guidewire lumen 310 to receive a guidewire therethrough. As illustrated, actuator tube 300 may be a dual lumen microcatheter having both guidewire lumen 310 and lumen 306 for receiving actuation wire 208 there through. Guidewire lumen 310 may extend more distally in the shaft than actuation lumen 306 for actuation wire 208 in actuator tube 300.

Actuator tube 300 may include hemostasis valve 312 at the proximal end to permit insertion of additional interventional devices (e.g., guidewire, actuation wire) into a lumen(s) of actuator tube 300 and to close off proximal end of lumens 306 and/or 310 when hemostasis valve 312 is closed.

Catheter 100 further may include intermediate tube 400, which is sized and shaped to be disposed within elongated tube 200. Intermediate tube 400 includes proximal region 402, distal region 404, and lumen 406 extending therebetween. Lumen 406 is sized and shaped to receive actuator tube 300 therethrough. Intermediate tube 400 may have a length less than actuator tube 300, but longer than the shaft of elongated tube 200, although actuation wire 208 may be longer than intermediate tube 400.

Intermediate tube 400 is slidably disposed within elongated tube 200 and may be used to reinforce the expandable section of elongated tube 200 during an intervention, such as removing an obstruction. For example, the distal end of intermediate tube 200 may be positioned proximally to the distal end of elongated tube 200 during delivery so as to maintain the low profile of catheter 200. Once suitable positioning is achieved in proximity to the target site (e.g., obstruction) in the blood vessel and elongated tube 200 has been transitioned to the expanded state, intermediate tube 400 may be advanced distally within elongated tube 200 to reinforce elongated tube 200 for the intervention, e.g., removal of the obstruction. Advantageously, the inner lubricious coating applied on the expandable section of elongated tube 200 described herein facilitates easy slidability of intermediate tube 300 within the lumen of elongated tube 200.

Proximal region 402 may be formed of a different material than distal region 404. For example, proximal region 402 may include shaft 408 formed from a polymer known in the art of catheter design to provide flexibility. Distal region 404 may include a coil 410 (e.g., biocompatible metal such as nitinol or stainless steel) having a biocompatible coating (e.g., PTFE). Coil 410 may be tightly wound such that adjacent turns in the coil contact one another. The diameter of intermediate tube 400 may be fixed such that intermediate tube 400 is not expandable. Intermediate tube 400 may include hemostasis valve 412 at the proximal end to permit insertion of additional interventional devices (e.g., actuator tube 300) into lumen 406 of intermediate tube 400 and to close off the proximal end of lumen 406 when hemostasis valve 412 is closed. One or more additional valves 414 may be connected to the proximal end of intermediate catheter 400, for example, to permit coupling to a vacuum source for aspiration of the obstruction in the blood vessel via catheter 100.

Referring now to FIGS. 1B and 1C, catheter 100 is shown, respectively, in a collapsed state and an expanded state. Actuator tube 300 is actuated to cause catheter 100 to transition from the collapsed state to the expanded state, and then may be further actuated cause catheter 100 to transition from the expanded state to the collapsed state. Actuator tube 300 may be moved in one direction (e.g., proximally) relative to actuation wire 208 to actuate the transition from collapsed to expanded and may be moved in another direction (e.g., distally) relative to actuation wire 208 to actuate the transition from expanded to collapsed. For example, actuator tube 300 may be unlocked by the clinician and moved proximally while elongated shaft 200 and intermediate tube 400 remain in place to cause distal region 204 (e.g., braid 216) of elongated shaft to expand. As such, elongated tube 200 is collapsible via longitudinal force at the distal end of elongated tube 200. As shown, elongated tube 200 is longer in the collapsed state than in the expanded state.

In this manner, the braided distal end of catheter 100 may be collapsed by being elongated. Braid elongation may be generally achieved by applying a longitudinal force to the distal end of the braided section. Conversely, the braid expansion may be achieved by releasing the longitudinal force. Advantageously, the distal end of elongated tube 200 may be held in place during expansion to prevent the catheter tip from jumping back upon the release of force. The self-collapsing mechanism allows force to be applied to the distal end of the braid as well as holding the distal end in place during expansion. As further advantages, these mechanisms allow advancing actuator tube 300 (e.g., a microcatheter) towards the distal end of elongated tube 200 to taper the diameter of the distal lumen and using actuator tube 300 to subsequently elongate braid 216 to narrow/collapse the entire distal braided section. As such, braid 216 may be collapsed onto actuator tube 300 to provide the sizing and flexibility of a microcatheter for delivery and removal of catheter 100.

In some embodiments, catheter 100 is designed to be inserted into the femoral artery of an adult human patient and to be navigated to the brain, for example to the middle cerebral artery, of the patient. Thus, the length of the catheter may be such that the catheter at least extends from the femoral artery of an adult human patient to the brain, in particular to the middle cerebral artery, of the same patient, to outside the patient for manipulation at the proximal end by the clinician. Depending on the application (e.g. in animals or humans, in children, female or male adults, etc.), catheter 100 may have an overall length of at least 30 cm, or at least 40 cm. For the use in humans, in particular in adult humans, the overall length of the catheter may be in a range between 100 cm and 200 cm, or in a range between 130 cm and 180 cm, for example.

The sizing of catheter 100 may be optimized for navigation in the neurovasculature. For example, the outer diameter of the shape-changing section distal section (e.g., the braided section) of elongated tube may be 0.5-6 mm in the collapsed state and 1-10 mm in the expanded state. In addition, the length of the shape-changing section distal section of elongated tube may be at least 10 cm from the distal end of elongated tube 200, such as 10-25 cm total, and may reduce about 5-25% in length when transitioned to the expanded state as compared to the collapsed state. In some embodiments, the diameter at the distal region of actuator tube 300 may be 0.4-2 mm for the dual lumen microcatheter configuration. The outer diameter of intermediate tube 400 may be 1-10 mm with an inner diameter of 0.8-8 mm. The length of the coiled section of intermediate tube 400 may extend at least 10 cm from the distal end of intermediate tube 400.

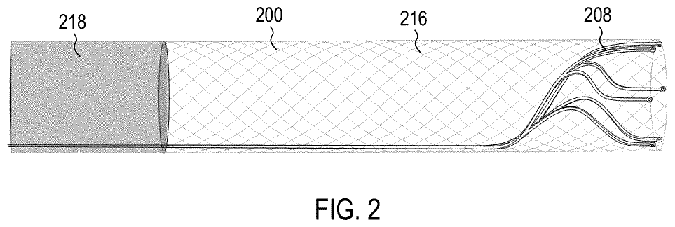

Referring now to FIG. 2, a view of an exemplary distal region of elongated tube 200 is shown. Elongated tube 200 may include braid 216 at the distal end and shaft 218 coupled to braid 216 more proximally. Actuation wire 208 is shown to facilitate self-collapse/expansion of the distal end of elongated tube 200 (e.g., the entire braided section). In some embodiments, the self-collapse/expansion mechanism (actuation wire 208) is mounted to the distal end of the braided structure to facilitate a reduction in the braid diameter by enabling the clinician to both collapse the distal braid and to elongate the braid by applying a force longitudinally to the distal end of the braid.

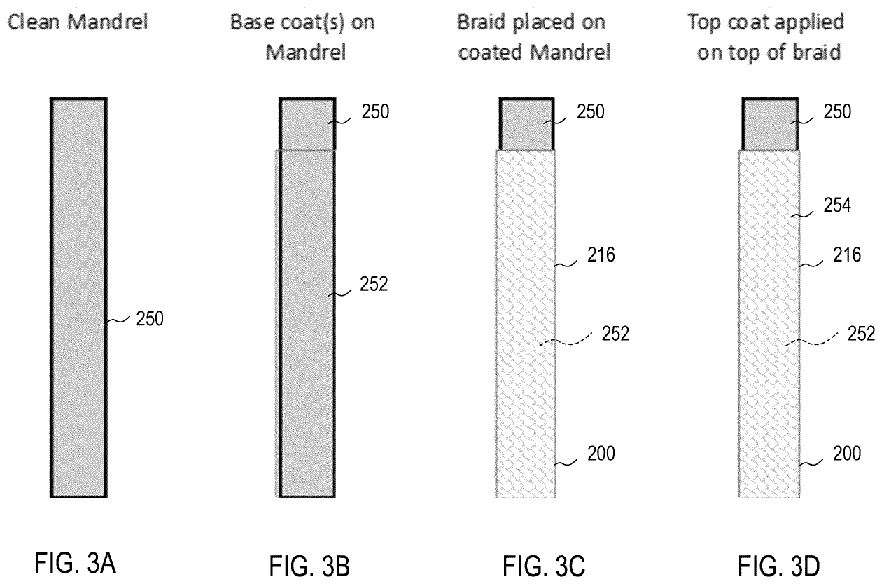

Referring now to FIGS. 3A-3D, an exemplary process is illustrated for applying an inner coating and an outer coating to an expandable portion of a catheter, such as elongated tube 200 of catheter 100 of FIGS. 1A-1C. It is understood that the steps set forth in FIGS. 3A-3D may apply to either all of or only a portion of the elongated tube (e.g., distal section). For example, the steps in FIGS. 3A-3D may only be applied to the distal 50 cm of the distal portion of the elongated tube. In one example, the steps in FIGS. 3A-3D may be applied to a portion of a distal portion of proximal section 202 and the entirety of distal region 204 of FIG. 1A. Some or all of the operations of the process flow may be optional and may be performed in a different order.

As shown in FIG. 3A, mandrel 250 is provided. Mandrel 250 is used for the sandwiching of the braid with the coatings. Mandrel 250 may be a PTFE coated stainless steel mandrel.

In FIG. 3B, base coat(s) is (are) applied to mandrel 250. This coating defines inner coating 252 to be applied to the inner surface of the expandable portion of the catheter (e.g., the braid). Inner coating 252 may be comprised of several layers of material.

In order to create a low friction coating on the inner lumen of the expandable portion of the catheter, the first base coat layer(s) of inner coating 252 is preferably formed from a lubricious material. This lubricious coating can be formed from materials such as hydrophilic polyurethanes (e.g., the Hydromed and Hydroslip family of materials from Advansource Biomaterials). Subsequent layer(s) of inner coating 252 can be selected so as to form a mechanically supporting layer for the lubricous layer. In addition, to improve the frictional behavior of the inner lumen of the expandable portion of the catheter, the mechanically supporting layer(s) can be formed from a material (e.g., polyurethane) with a higher hardness than the innermost layer(s) of inner coating 252 in order to mitigate the risk of any tackiness. As such, the innermost layer(s) of inner coating 252 may have different characteristics than the outermost layer(s) of inner coating 252.

Inner coating 252 may be formed of multiple layers, e.g., multiple layers of polymers such as polyurethane. For example, an exemplary coating configuration for the base coat involves a layered deposition on the mandrel. There may be four layers formed by: Layer 1: 5% w/w (Hydromed D4: Hydroslip C 70:30) in IPA/Water 95/5; Layer 2: 5% w/w (Hydromed D4: Hydroslip C 70:30) in IPA/Water 95/5; Layer 3: 3% w/w Tecoflex 93a in THF; Layer 4: 3% w/w Tecoflex 93a in THF.

Each of the layers of inner coating 252 are dipped at a first rate of speed (e.g., 1000 mm/min) and withdrawn at a second rate of speed (e.g., 350 mm/min) and are allowed to dry for a layer-drying time period (e.g., 2 minutes). Following application of these base layers, inner coating 252 is dried by heating at a temperature (e.g., at 70° C.) for a coating-drying time period (e.g., 20 minutes).

After inner coating 252 is applied to mandrel 250, inner coating 252 is permitted to dry. Next, as shown in FIG. 3C, once the coating has been dried, the expandable portion of the catheter 200 (e.g., braid 216 which can be a nitinol braid) is placed on mandrel 250.

Next, as shown in FIG. 3D, outer coating 254 is applied to the expandable portion of the catheter 200 (e.g., braid 216). Outer coating 254 may be formed of multiple layers, e.g., multiple layers of polymers such as polyurethane. The layer(s) of outer coating 254 preferably has different characteristics to the layer(s) of inner coating 252.

For example, the shore hardness of the layers of polyurethane of outer coating 254 can be relatively softer than the base layers of inner coating 252 so that the layers of outer coating 254 can be thicker to cover the braid wires, while at the same time not contributing excessively to the structure stiffness.

Outer coating 254 may be a top coat layer of 5% Tecoflex 80A. After application outer coating is allowed to dry for a drying time period (e.g., 2 minutes) and then further dried in an oven by heating at a temperature (e.g., 70° C.) for a time period (e.g., 20 minutes).

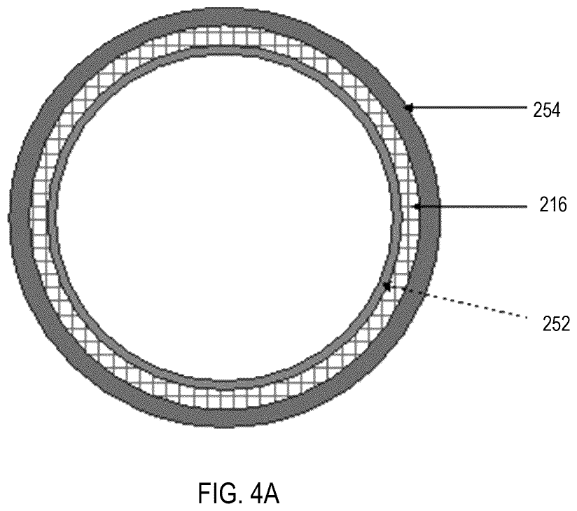

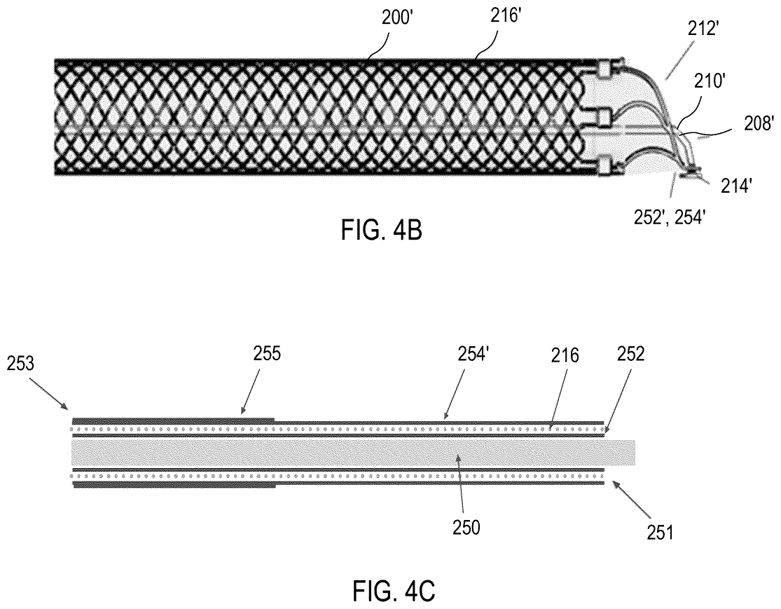

FIG. 4A illustrates a cross-sectional view of an expandable portion of a catheter (e.g., the braid) with inner and outer coatings thereon. As shown, braid 216 is sandwiched between inner coating 252 and outer coating 254. Advantageously, braid 216 with inner coating 252 and outer coating 254 is configured to transition from a contracted state to an expanded state at the target site in the blood vessel for performing an intervention, e.g., removal of an obstruction(s) from a blood vessel. Inner coating 252 defines a lubricious inner surface lumen such that an interior catheter, such as intermediate tube 400, can be slid into and out of the lumen of braid 216 while expanded for reinforcing the expanded braid.

Referring now to FIG. 4B, an alternative distal region of elongated tube 200′ is shown. Similar to elongated tube 200 described above, elongated tube 200′ includes braid 216′ and actuation wire 208′ to facilitate self-collapse/expansion of the distal end of elongated tube 200′ (e.g., the entire braided section), however, the plurality of struts 212′ of actuation wire 208′ are coupled to elongated shaft 210′ at articulation region 214′ at an area past the distal end of braid 216′. One or more lubricious coatings (e.g., inner and outer coatings 252′, 254′ described above) are also applied to the plurality of struts 212′ beyond the braided section. These lubricious coating(s) on the struts permits movement through the vasculature during advancement of the catheter to the target site without interference between the struts and the inner walls of the vasculature.

Referring now to FIG. 4C, a side view of an alternative expandable portion (e.g., expandable distal section) of the catheter including a braid and inner and outer coatings thereon is shown. The catheter is illustrated positioned on mandrel 250. Similar to the expandable portion of the catheter illustrated in FIG. 4A, braid 216 is positioned on mandrel 250 with inner coating 252 between mandrel 250 and braid 216. Outer coating 254′ may be positioned on an exterior of braid 216 and may be similar to outer coating 254 of FIG. 4A.

As shown in FIG. 4C, proximal region 253 of the catheter may include enhanced region 255 of outer coating 254 which may be thicker (e.g., may have a larger outer diameter) than outer coating 254′ at distal region 251 of the catheter. To achieve enhanced region 255, outer coating 254′ may be applied evenly across an exterior of braid 216 and mandrel 250 may be manipulated such that distal region 251 is positioned higher than proximal region 253, causing gravity to guide excess outer coating 254′ at distal region 251 towards proximal region 253, resulting enhanced region 255 with thicker outer coating 254′ near proximal region 253 than distal region 251. Alternatively, or additionally, mandrel 250 excess outer coating 254′ may be guided toward proximal region 253 via a fan or other force. In another example, after outer coating 254′ is evenly applied across exterior braid 216, a second layer of outer coating may be applied to region 255 resulting in outer coating 254′ in region 255 to be thicker near proximal region 253 of the catheter than near distal region 251 of the catheter.

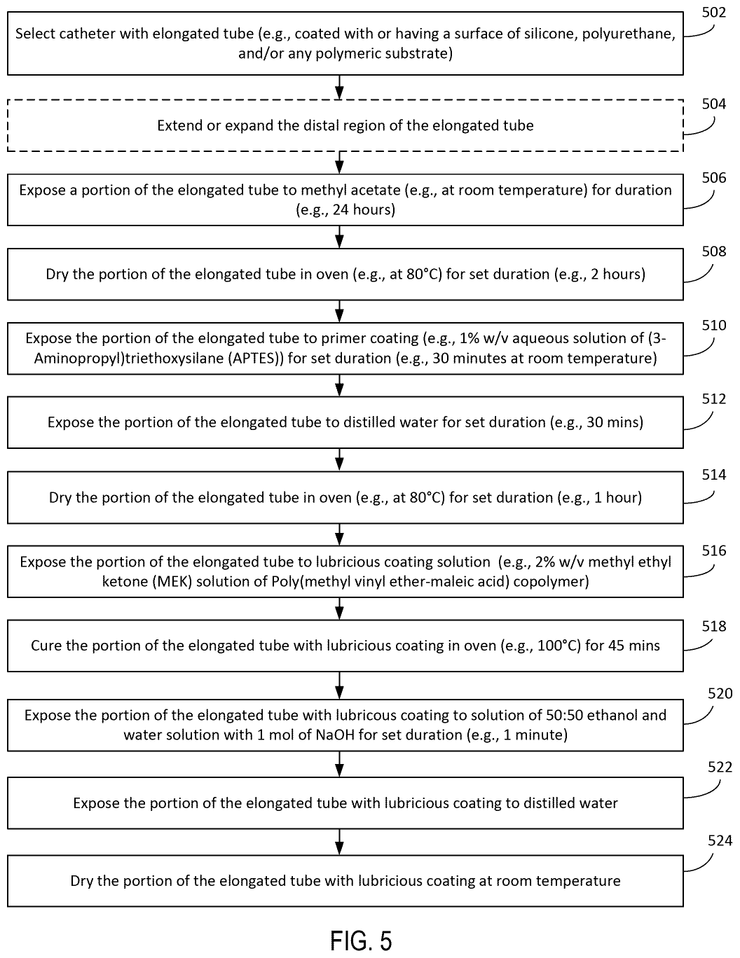

Referring now to FIG. 5, a process flow is provided in accordance with another embodiment for applying a preliminary solution, a primer coating, and lubricious coating to an elongated tube of a catheter, such as elongated tube 200 of catheter 100 of FIGS. 1A-IC. It is understood that the steps set forth in FIG. 5 may apply to either all of or only a portion of the elongated tube (e.g., distal section). For example, the steps in FIG. 5 may only be applied to the distal 50 cm of the distal portion of the elongated tube. In one example, the steps in FIG. 5 may be applied to a portion of a distal portion of proximal section 202 and the entirety of distal region 204 of FIG. 1A. Some or all of the operations of the process flow may be optional and may be performed in a different order.

At block 502, a catheter may be selected. For example the catheter may be catheter 100 of FIGS. 1A-1C and may include elongated tube 200 with proximal region 202 and distal region 204, which may include a braid (e.g., braid 216) for example. Some or all of the elongated tube may be coated with or otherwise have an inner surface and/or an outer surface of silicone, polyurethane, and/or any other polymeric substrate.

At optional block 504, the distal region of elongated tube (e.g., distal region 204 of FIGS. 1A-1C) may be transitioned into an extended or expanded state. For example, the elongated tube may be transitioned from a collapsed state to an expanded state and may be held at this position for the remaining steps. Alternatively, the distal region may be in any other state such as a collapsed state or neutral or resting state. In another example, the distal region of the elongated tube may be extended to a maximum length of the elongated tube and maintained in this stretched state.

At block 506, a portion (e.g., distal portion) of the elongated tube may be exposed to methyl acetate at room temperature. For example the portion may be dipped into the solution such that an inner surface and an outer surface of the elongated tube are expose simultaneously. The elongated tube may be exposed for a duration of 24 hours or anywhere within the range of 22-26 hours. The methyl acetate solution may remove mobile low molecular weight silicone molecules, for example,

At block 508, the portion of the elongated tube may be dried. For example, all or a portion of the elongated tube may be placed in an oven (e.g., convection oven). The oven may be set at a temperature of 80° C. or anywhere between 70° C. and 90° C. The elongated tube may be placed in the oven for 2 hours or any time between 90 minutes and 150 minutes.

At block 510, the portion of the elongated tube may be exposed to (e.g., dipped into) a primer coating. The primer coating may be an aqueous solution of (3-Aminopropyl)triethoxysilane (APTES)). For example, the primer coating may include 1% APTES weight-by-volume. The portion of the elongated tube may be positioned in the primer coating 30 minutes or any period of time between 20 and 40 minutes. The primer coating may be room temperature for the duration of exposure.

At block 512, the portion of the elongated tube may be exposed to (e.g., dipped into) water (e.g., distilled water) for 30 minutes or otherwise for a period of time between 20 minutes and 40 minutes. At block 514, the portion of the elongated tube may be placed into an oven (e.g. convection oven). The oven may be set at a temperature of 80° C. or anywhere between 70° C. and 90° C. The elongated tube may be placed in the oven for 1 hour or any time between 45 minutes and 75 minutes.

At block 516, the portion of the elongated tube may be exposed to (e.g., dipped into) a lubricious coating solution. For example, the lubricious coating solution may be a methyl ethyl ketone (MEK) solution of poly(methyl vinyl ether-maleic acid) copolymer. The solution may include 2% MEK volume-by-weight. In one example, the solution may be or may include Gantrez™ AN-169. It is understood that other suitable lubricious coatings may be additionally or alternatively used and/or applied to the portion of the elongated tube.

At block 518, the portion of the elongated tube may be placed into an oven (e.g. convection oven) to cure the lubricious coating on the outer and/or inner surfaces of the elongated tube. The oven may be set at a temperature of 100° C. or anywhere between 90° C. and 110° C. The elongated tube may be placed in the oven for 1 hour or any time between 45 minutes and 75 minutes. Alternatively, or additionally, the lubricious coating may be cured using ultraviolet (UV) light. For example, the lubricious coating solution may include suitable photo-initiators, which may respond to the UV light to cure the lubricious coating on the elongated tube. UV may be applied to the outer surface and the inner surface separately to cure the outer surface and the inner surface.

At block 520, the portion of the elongated tube with the lubricious coating may be exposed to (e.g., dipped into) a solution of equal parts (e.g., 50:50) ethanol and water. The solution may include sodium hydroxide (NaOH). For example, the solution may include 1 mol of NaOH. The portion of the elongated tube may be exposed to the solution for 1 minute or for any time between 30 seconds and 90 seconds.

At block 522, the portion of the elongated tube with the lubricious coating may be exposed to (e.g., dipped into) water. At block 524, the portion of the elongated tube with the lubricious coating may be air dried (e.g., at room temperature). It is understood that one or more blocks of FIG. 5 may be optional. For example, blocks 504, 508, 512, 514, 522 and/or 524 may be optional. It is further understood that different ranges of temperatures and time periods than those provided with respect to FIG. 5 may be used to achieve similar results.

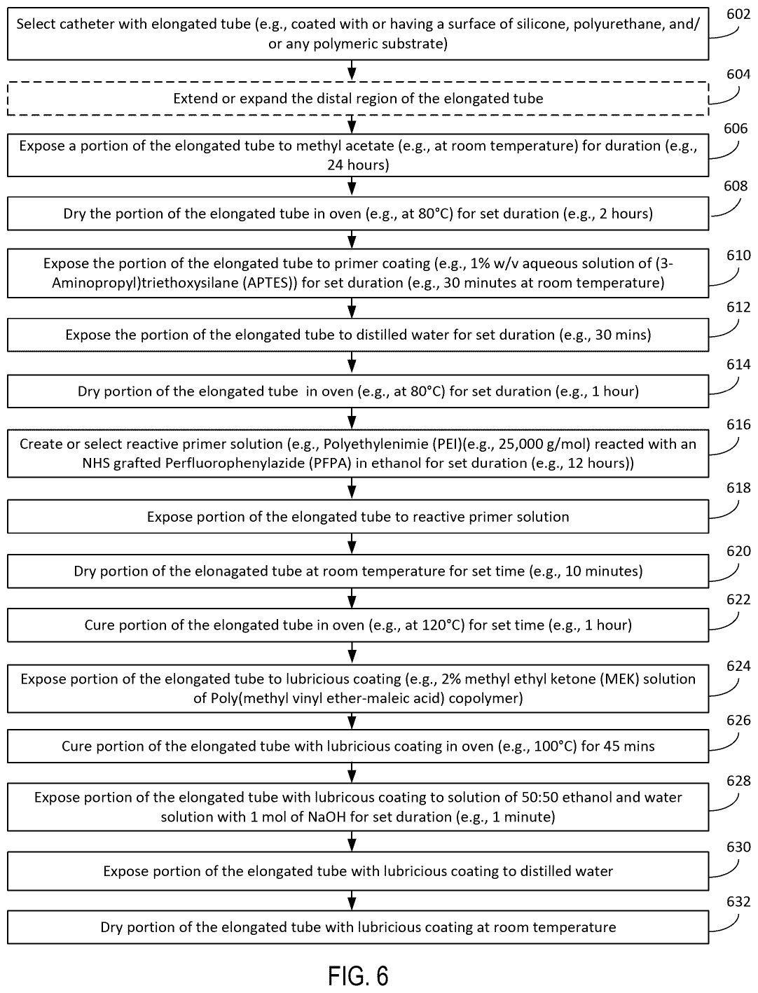

Referring now to FIG. 6, a process flow for applying a preliminary solution, a primer coating, a reactive primer coating, and a lubricious coating to an elongated tube of a catheter, such as elongated tube 200 of catheter 100 of FIGS. 1A-1C, for example, is depicted. It is understood that the steps set forth in FIG. 6 may apply to either all of or only a portion of the elongated tube (e.g., distal end). For example, the steps in FIG. 6 may only be applied to the distal 50 cm of the distal portion of the elongated tube. The steps in FIG. 6 may be applied to only a portion of a distal portion of proximal section 202 and the entirety of distal region 204 of FIG. 1A. Some or all of the operations of the process flow may be optional and may be performed in a different order.

Blocks 602-614 of FIG. 6 may be the same as or similar to blocks 502-514 of FIG. 5, respectively. After block 614, at block 616 a reactive primer solution may be created, mixed, and/or selected. The reactive primer solution may include an ethanol solution with polyethylenimie (PEI) reacted with an N-Hydroxysuccinimide (NHS) grafted Perfluorophenylazide (PFPA) for a period of 12 hours or any time between 10 and 14 hours. The solution may include 25,000 g/mol of PEI. The reactive primer solution may include a ratio of PEI to PFPA between 4:1 to 10:1 molar equivalent.

At block 618, a portion of the elongated tube may be exposed to (e.g., dipped into) the reactive primer solution created, mixed, and/or selected at block 616. At block 620, the portion of the elongated tube with the reactive primer may be air dried (e.g., at room temperature). At block 622, the portion of the elongated tube may be placed into an oven (e.g. convection oven) to cure the reactive primer on the outer and/or inner surfaces of the elongated tube. The oven may be set at a temperature of 120° F. or anywhere between 110° F. and 130° F. The elongated tube may be placed in the oven for 1 hour or any time between 45 minutes and 75 minutes. Alternatively, or additionally, the reactive primer may be cured using ultraviolet (UV) light. For example, the reactive primer may include photo-initiators that may be activated via the UV light.

Block 624 may be initiated after block 622. Blocks 624-632 may be the same as or similar to blocks 516-525 of FIG. 5. It is understood that one or more blocks of FIG. 6 may be optional. For example, blocks 604, 612, 614, 620, 628, 630 and/or 632 may be optional. It is further understood that different ranges of temperatures and/or time periods than those provided with respect to FIG. 6 may be used to achieve similar results.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A catheter system for advancement in a vasculature to a target site in a blood vessel, the catheter system comprising:
an elongated tube comprising a proximal section and an expandable distal section, the expandable distal section comprising an inner coating forming an inner lubricious surface and an outer coating, the expandable distal section with the inner coating and the outer coating configured to transition from a contracted state to an expanded state at the target site in the blood vessel for performing an intervention; and
an inner elongated tube configured to be advanced into the expandable distal section in the expanded state via the inner lubricious surface.

2. The catheter system of claim 1, further comprising an actuation tube configured to be disposed within a lumen of the inner elongated tube, the actuation tube configured to cause the expandable distal section to transition from the contracted state to the expanded state.

3. The catheter system of claim 1, wherein the inner coating comprises an innermost lubricious layer that forms the inner lubricious surface and a supporting layer having a higher hardness than the innermost lubricious layer.

4. The catheter system of claim 3, wherein the innermost lubricious layer and the supporting layer are each formed of polyurethane.

5. The catheter system of claim 1, wherein the inner coating comprises four layers.

6. The catheter system of claim 1, wherein the expandable distal section comprises a nitinol braid.

7. The catheter system of claim 1, wherein the outer coating is softer than the inner coating.

8. The catheter system of claim 1, wherein the expandable distal section comprises a proximal region and a distal region and wherein the outer coating is thicker near the proximal region than the distal region.

9. The catheter system of claim 1, wherein the outer coating comprises polyurethane.

10. The catheter system of claim 1, wherein the inner coating comprises multiple layers of polyurethane and the outer coating comprises multiple layers of polyurethane.

11. The catheter system of claim 1, wherein the layers of polyurethane of the outer coating are softer than the layers of polyurethane of the inner coating.

12. The catheter system of claim 1, wherein the inner coating is applied to a mandrel, the expandable distal section is applied on the inner coating on the mandrel, and the outer coating is applied on the expandable distal section while on the mandrel.

13. The catheter system of claim 1, further comprising a plurality of struts positioned distal to the expandable distal section and coupled to the expandable distal section to cause the expandable distal section to transition from the contracted state to the expanded state,
wherein the plurality of struts are coated with the inner coating, the outer coating, or both.

14. A method for applying a lubricious coating to a neurovascular catheter, the method comprising:
selecting a catheter comprising an elongated tube comprising a proximal section and a braided section at a distal end, the elongated tube having an inner surface and an outer surface each comprising silicone or polyurethane, the catheter adapted to remove an obstruction from a blood vessel;
exposing a distal portion of the catheter comprising the braided section and at least part of the proximal section to a first solution comprising methyl acetate for a first duration;
exposing, after exposing the distal portion of the catheter to the first solution, the distal portion of the catheter to a second solution comprising (3-Aminopropyl) triethoxysilane (APTES) for a second duration;
exposing, after exposing the distal portion of the catheter to the second solution, the distal portion of the catheter to a lubricious coating solution comprising a poly(methyl vinyl ether-maleic acid) copolymer to apply a lubricous coating to the distal portion of the catheter; and
heating, after exposing the distal portion of the catheter to the lubricious coating solution, the distal portion of the catheter at a first temperature for a third duration.

15. The method of claim 14, wherein the first duration is between 22 and 26 hours, the second duration is between 20 and 40 minutes, and the third duration is between 35 and 55 minutes and the first temperature is between 90° F. to 110° C.

16. The method of claim 14, wherein the second solution is an aqueous solution comprising 1% weight-by-volume of (3-Aminopropyl)triethoxysilane and, wherein the lubricious coating solution comprises 2% weight-by-volume of methyl ethyl ketone (MEK).

17. The method of claim 14, further comprising, after heating the distal portion of the catheter, exposing at least the distal portion of the catheter to a fourth solution comprising equal portions water and ethanol.

18. A method for applying a lubricious coating to a neurovascular catheter, the method comprising:
selecting a catheter comprising an elongated tube comprising a proximal section and a braided section at a distal end, the elongated tube having an inner surface and an outer surface each comprising silicone or polyurethane, the catheter adapted to remove an obstruction from a blood vessel;
exposing a distal portion of the catheter comprising the braided section and at least part of the proximal section to a first solution comprising methyl acetate for a first duration;
exposing, after exposing the distal portion of the catheter to the first solution, the distal portion of the catheter to a second solution comprising (3-Aminopropyl) triethoxysilane (APTES) for a second duration;
selecting a reactive primer solution comprising Polyethylenimie (PEI) reacted with an N-Hydroxysuccinimide (NHS) grafted Perfluorophenylazide (PFPA) in ethanol;
exposing, after exposing the distal portion of the catheter to the second solution, the distal portion of catheter to the reactive primer solution; and
exposing, after exposing the distal portion of the catheter to the primer coating, the distal portion of catheter to a lubricious coating solution comprising a poly(methyl vinyl ether-maleic acid) copolymer to apply a lubricous coating to the distal end of the catheter.

19. The method of claim 18, wherein the lubricious coating solution comprises 25,000 g/mol of branched Polyethylenimie (PEI) or 2% weight-by-volume of methyl ethyl ketone (MEK).

20. The method of claim 18, wherein the reactive primer solution comprises a ratio of PEI to PFPA between 4:1 to 10:1 and the second solution is an aqueous solution comprising 1% weight-by-volume of (3-Aminopropyl)triethoxysilane.
